(19) United States
(12) Patent Application Publication
(10) Pub. No.: US 2018/0163266 A1
(43) Pub. Date: Jun. 14, 2018

(54) NUCLEOTIDE SENSING DEVICE HAVING A NANOPORE FORMED IN AN INORGANIC MATERIAL

(71) Applicant: QUALCOMM Incorporated, San Diego, CA (US)

(72) Inventors: Tallis Young CHANG, San Diego, CA (US); Yaoling PAN, San Diego, CA (US); Yong Ju LEE, San Diego, CA (US)

(21) Appl. No.: 15/373,007

(22) Filed: Dec. 8, 2016

(51) Int. Cl. C12Q 1/68; G01N 27/447; G01N 33/487; G03F 7/20; H01L 27/092

(52) U.S. Cl. CPC C12Q 1/6869; G01N 27/44791; H01L 27/092; G03F 7/70; G01N 33/48721

(57) ABSTRACT

Methods and apparatuses for sensing nucleotides are disclosed. A related nucleotide sensing device may include an insulator having an electrode well and a separation layer attached to the insulator, the separation layer including a film and a shell layer. The film may have a hole, the hole having a first diameter. The shell layer may be disposed on a surface of the film, and at least a portion of the shell layer may be disposed within the hole. The separation layer may be formed of inorganic material and may comprise a nanopore. The nanopore may permit fluid communication with the electrode well across the separation layer. The nanopore may be disposed within the hole and may have a second diameter smaller than the first diameter.

### INTRODUCTION

Aspects of this disclosure relate generally to sensing devices for nucleotides, and more particularly to methods and apparatuses for sensing nucleotides using a nucleotide sensing device having a nanopore formed in an inorganic material.

Ribonucleic acid (RNA) and deoxyribonucleic acid (DNA), sometimes referred to as the “blueprint of life”, are molecules that stores biological information. RNA, for example, includes a strand of biopolymer that includes a plurality of nucleotides. The structure of DNA, famously discovered by James Watson and Francis Crick, consists of two strands of biopolymer, coiled around one another to form a double helix. Each strand in DNA is a polynucleotide that includes a plurality of nucleotides, for example, cytosine (“C”), guanine (“G”), adenine (“A”), and thymine (“T”). Each nucleotide in a first strand of DNA may be bonded to a paired nucleotide in the second strand, thereby forming a base pair. Generally, cytosine and guanine are paired to form a “G-C” or “C-G” base pair, and adenine and thymine are paired to form an “A-T” or “T-A” base pair.

Although the structure of RNA and DNA is now known, new methods for analyzing individual molecules are still being developed. Generally, the analysis includes ‘reading’ the nucleotide sequence of a particular strand of RNA or DNA (referred to herein as a “nucleotide strand”). In one method, known as nanopore sequencing, a nanopore is immersed in a conductive fluid, and a voltage is applied across the nanopore. As a result, ions are conducted through the nanopore, thereby generating a measurable electric current. A nucleotide strand is then transmitted through a nanopore, one nucleotide at a time. The presence of a nucleotide within the nanopore disrupts the conduction of the ions, thereby causing a change in the electric current. Moreover, the change in electrical current due to a particular nucleotide differs from the change in electrical current due to other nucleotides. Accordingly, an entire nucleotide strand can be transmitted through the nanopore and each nucleotide in the strand can be identified based on the change in current. Over time, the changes in electric current result in a nucleotide sensing signal reflecting the particular nucleotides in a particular nucleotide strand.

As nanopore sequencing improves, new challenges are presented. For example, a capacitance may arise within the nucleotide sensing device. The capacitance may limit the bandwidth of the nucleotide sensing signal by reducing the maximum cutoff frequency. The capacitance may also increase a noise component of the nucleotide sensing signal. As a result, new technologies are needed for improving the nucleotide sensing signal of nanopore-based nucleotide sensing devices.

### SUMMARY

The following summary is an overview provided solely to aid in the description of various aspects of the disclosure and is provided solely for illustration of the aspects and not limitation thereof.

In one example, a nucleotide sensing device is disclosed. The nucleotide sensing device may include, for example, an insulator having an electrode well, and a separation layer attached to the insulator, the separation layer including a film and a shell layer, wherein the film has a hole, the hole having a first diameter, the shell layer is disposed on a surface of the film, and at least a portion of the shell layer is disposed within the hole, and the separation layer comprises a nanopore, the nanopore permitting fluid communication across the separation layer, the nanopore being disposed within the hole and having a second diameter smaller than the first diameter.

In another example, a method of forming a nucleotide sensing device is disclosed. The method may include, for example, providing a film, the film being provided on an oxide layer, etching a hole in the film, the hole in the film having a first diameter, forming a separation layer by disposing a shell layer on a surface of the film, wherein the separation layer comprises a nanopore, the nanopore having a second diameter smaller than the first diameter, and attaching the separation layer to an insulator having an electrode well.

In yet another example, a nucleotide sensing device is disclosed. The nucleotide sensing device may include, for example, means for insulating, and means for separating attached to the means for insulating, the means for separating including means for supporting the means for separating and means for covering the means for supporting, wherein the means for supporting has a hole, the hole having a first diameter, the means for covering is disposed on a surface of the means for supporting, and at least a portion of the means for covering is disposed within the hole, and the means for separating comprises means for permitting fluid communication across the means for separating, the means for permitting fluid communication being disposed within the hole and having a second diameter smaller than the first diameter.

### BRIEF DESCRIPTION OF THE DRAWINGS

The accompanying drawings are presented to aid in the description of various aspects of the disclosure and are provided solely for illustration of the aspects and not limitation thereof.

FIG. 1 generally illustrates a nucleotide sensor array in accordance with aspects of the disclosure.

FIG. 2A generally illustrates a pair of nanopore-based nucleotide sensing devices in accordance with aspects of the disclosure.

FIG. 2B generally illustrates a detail of the biological pore of FIG. 2A in accordance with an aspect of the disclosure.

FIG. 3 generally illustrates another pair of nanopore-based nucleotide sensing devices in accordance with aspects of the disclosure.

FIG. 4 generally illustrates a method for forming a nucleotide sensing device such as the nucleotide sensing device of FIG. 2A and/or the nucleotide sensing device of FIG. 3.

FIG. 5A generally illustrates the nucleotide sensing device in a first stage of fabrication.

FIG. 5B generally illustrates the nucleotide sensing device in a second stage of fabrication.

FIG. 5C generally illustrates the nucleotide sensing device in a third stage of fabrication.

FIG. 5D generally illustrates the nucleotide sensing device in a fourth stage of fabrication.

FIG. 5E generally illustrates the nucleotide sensing device in a fifth stage of fabrication.

FIG. 5F generally illustrates the nucleotide sensing device in a sixth stage of fabrication.

FIG. 5G generally illustrates the nucleotide sensing device in a seventh stage of fabrication.

FIG. 5H generally illustrates the nucleotide sensing device in an eighth stage of fabrication.

FIG. 6A generally illustrates another nucleotide sensing device in a stage of fabrication analogous to the second stage of fabrication depicted in FIG. 5B.

FIG. 6B generally illustrates the nucleotide sensing device depicted in FIG. 6A in a stage of fabrication analogous to the eighth stage of fabrication depicted in FIG. 5H.

### DETAILED DESCRIPTION

The present disclosure relates generally to a method and apparatus for increasing the lifespan and manufacturability of a nanopore DNA sensing device.

More specific aspects of the disclosure are provided in the following description and related drawings directed to various examples provided for illustration purposes. Alternate aspects may be devised without departing from the scope of the disclosure. Additionally, well-known aspects of the disclosure may not be described in detail or may be omitted so as not to obscure more relevant details.

It will be understood that terms such as “top” and “bottom”, “left” and “right”, “vertical” and “horizontal”, etc., are relative terms used strictly in relation to one another, and do not express or imply any relation with respect to gravity, a manufacturing device used to manufacture the components described herein, or to some other device to which the components described herein are coupled, mounted, etc.

Those of skill in the art will appreciate that the information and signals described below may be represented using any of a variety of different technologies and techniques. For example, data, instructions, commands, information, signals, bits, symbols, and chips that may be referenced throughout the description below may be represented by voltages, currents, electromagnetic waves, magnetic fields or particles, optical fields or particles, or any combination thereof, depending in part on the particular application, in part on the desired design, in part on the corresponding technology, etc.

Further, many aspects are described in terms of sequences of actions to be performed by, for example, elements of a computing device. It will be recognized that various actions described herein can be performed by specific circuits (e.g., Application Specific Integrated Circuits (ASICs)), by program instructions being executed by one or more processors, or by a combination of both. In addition, for each of the aspects described herein, the corresponding form of any such aspect may be implemented as, for example, “logic configured to” perform the described action.

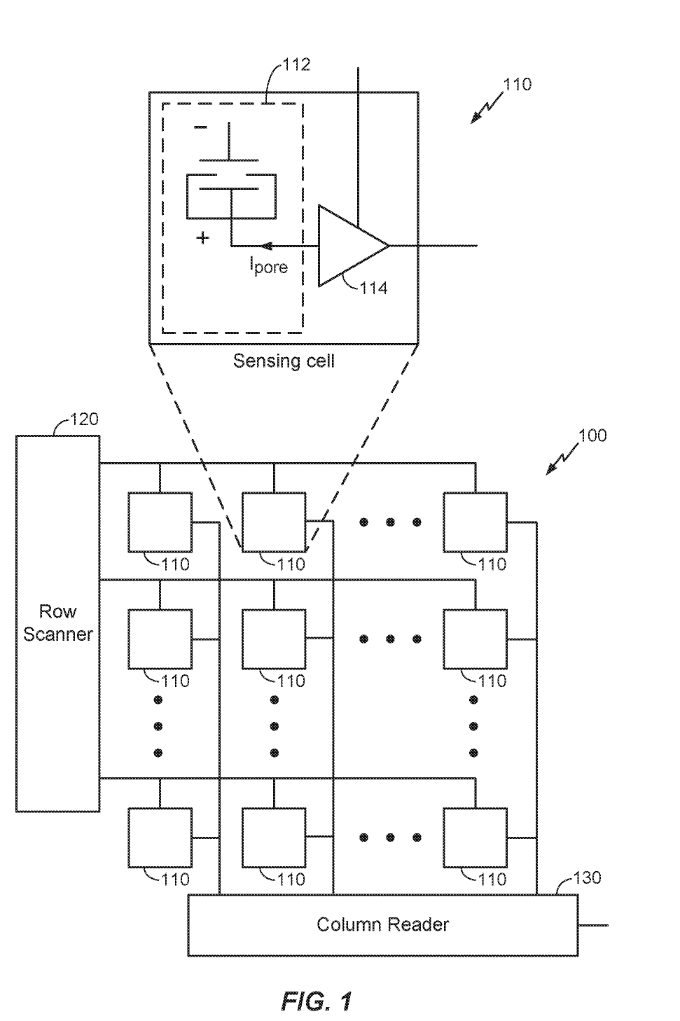

FIG. 1 generally illustrates a nucleotide sensor array 100 in accordance with aspects of the disclosure.

The nucleotide sensor array 100 may include a plurality of nucleotide sensing cells 110. The plurality of nucleotide sensing cells 110 may be arranged in rows and columns to form a grid pattern. The nucleotide sensor array 100 may further include a row scanner 120 and a column reader 130. The row scanner 120 and column reader 130 may facilitate the reading of a particular nucleotide sensing cell 110 from among the plurality of nucleotide sensing cells 110.

Each nucleotide sensing cell 110 may include a nucleotide sensing device 112 and an amplifier 114. As will be described in greater detail below, the nucleotide sensing device 112 may dynamically generate an electric current as a nucleotide strand is transmitted through a nanopore in the nucleotide sensing device 112. The nucleotide strand may comprise, for example, a strand of RNA, a single strand of DNA, or any other biopolymer. Over time, the changes in electric current result in a nucleotide sensing signal reflecting the particular nucleotides in a particular nucleotide strand. The amplifier 114 may facilitate amplification of the DNA sensing signal.

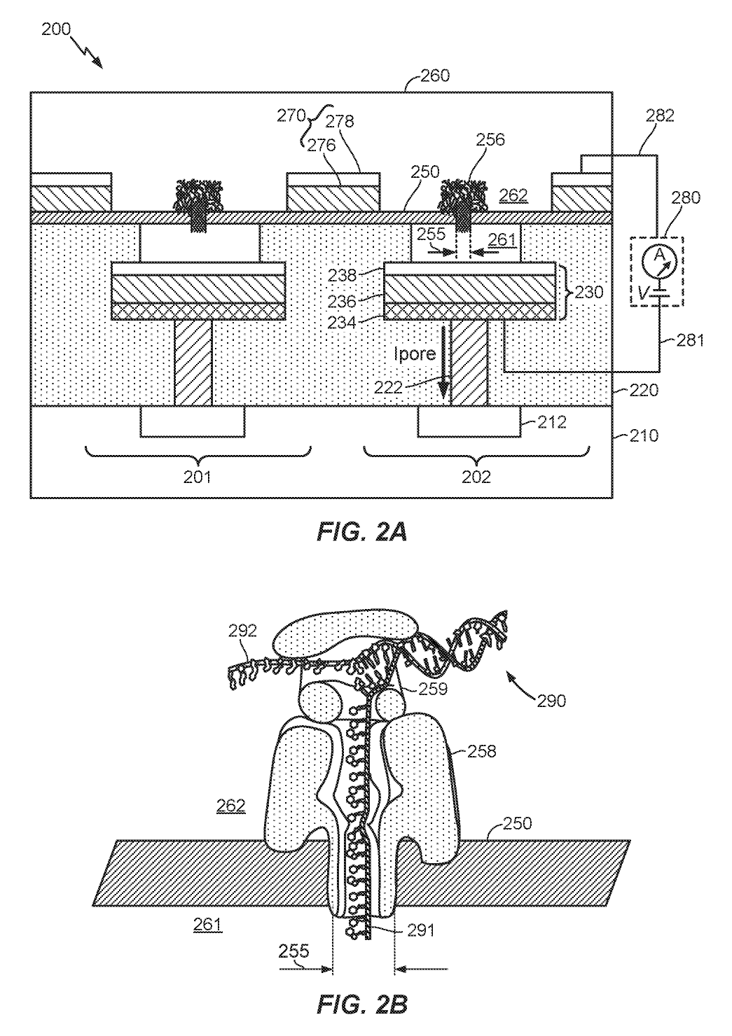

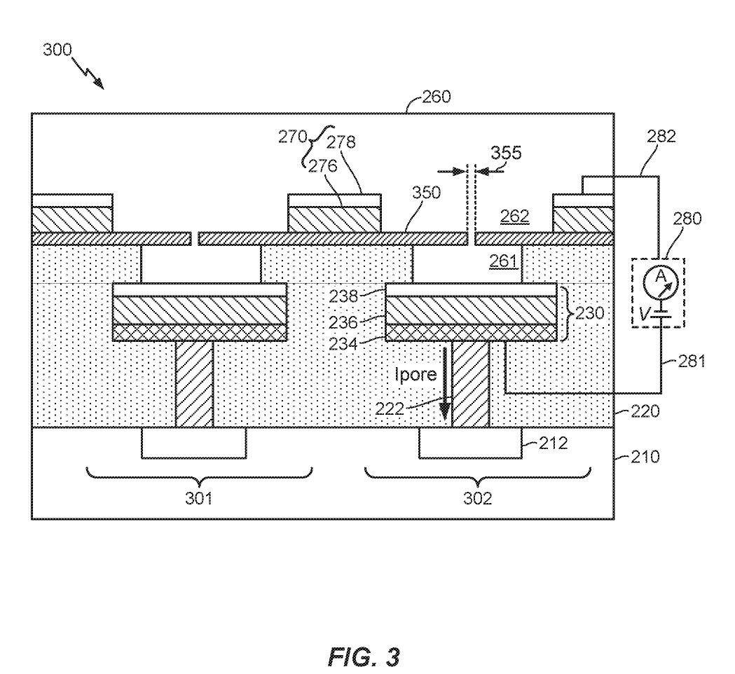

FIGS. 2A-2B and FIG. 3 generally illustrate nanopore-based nucleotide sensing devices having nanopores formed in a separation layer, the separation layer comprising inorganic materials. In previous approaches, the separation layer may have comprised organic materials, for example, a lipid bilayer. A biological nanopore may have been embedded in the lipid bilayer. However, the process of embedding the biological nanopore in the lipid bilayer can be costly and time-consuming, and the resulting nucleotide sensing device can be fragile. Accordingly, the techniques depicted in FIGS. 2A-2B and 3 may improve the cost and quality of nucleotide sensing devices. In particular, FIGS. 2A-2B depict an inorganic separation layer having a biological pore provided therein, whereas in FIG. 3, the biological pore is omitted from the inorganic separation layer.

FIG. 2A generally illustrates a pair 200 of nanopore-based nucleotide sensing devices 201, 202 in accordance with aspects of the disclosure. The nucleotide sensing devices 201, 202 may be analogous to the nucleotide sensing device 112 depicted in FIG. 1. The nucleotide sensing devices 201, 202 may be formed on a substrate 210 and at least partially within an insulator 220. As used herein, the term “insulator” refers to a component that is at least partially composed of one or more insulative subcomponents which are either electrically, chemically, and/or thermally insulative. The substrate 210 may include, for example, silicon (Si) and the insulator 220 may include, for example, silicon oxide (SiO₂), silicon mononitride (SiN), a hydrophilic material, any combination thereof, or any other suitable material(s). The nucleotide sensing devices 201, 202 may be disposed in adjacent DNA sensing cells analogous to the nucleotide sensing cells 110 depicted in FIG. 1. The nucleotide sensing devices 201, 202 depicted in FIG. 2 may be substantially similar to one another. Accordingly, the particular components of only one nanopore-based nucleotide sensing device will be described below.

The nucleotide sensing device 202 may include a semiconductor device 212 disposed on the substrate 210. The semiconductor device 212 may include, for example, a complementary metal-oxide-semiconductor (CMOS) circuit having one or more transistors. The semiconductor device 212 may be a component of an amplifier analogous to the amplifier 114 depicted in FIG. 1. The insulator 220 may include a via 222 in contact with the semiconductor device 212. The via 222 may include, for example, copper (Cu), tungsten (W), aluminum (Al), any combination thereof, or any other suitable material(s).

The nucleotide sensing device 202 further includes a first electrode 230 in contact with the via 222. The first electrode 230 may be disposed on or within the insulator 220. The first electrode 230 may include an adhesion/diffusion layer 234, a conductive layer 236, and a surface layer 238.

As an example, the adhesion/diffusion layer 234 may include a chromium (Cr) adhesion layer in contact with the via 222 and a gold (Au) diffusion layer between the conductive layer 236 and the Cr adhesion layer. Additionally or alternatively, the adhesion/diffusion layer 234 may include titanium nitride (TiN) and/or any other suitable material(s). The conductive layer 236 may include silver (Ag), however, it will be understood that any suitable material may be selected. The surface layer 238 may include silver chloride (AgCl), however, it will be understood that any suitable material(s) may be selected.

The nucleotide sensing device 202 depicted in FIG. 2A further includes a separation layer 250 and a chamber 260. The separation layer 250 may comprise a nanopore 255 having a biological pore 256 embedded therein. As will be discussed in greater detail below, the size of the nanopore 255 may be selected to accommodate the biological pore 256.

The chamber 260 may hold a conductive fluid therein. The conductive fluid may include, for example, one or more electrolytes, for example, chlorine electrolyte (Cl⁻), potassium electrolyte (K+), hydrogen electrolyte (H+), or any other suitable material. The conductive fluid within the chamber 260 may be divided by the separation layer 250 into a first subchamber 261 and a second subchamber 262.

The nucleotide sensing device 202 may further include a second electrode 270. The second electrode 270 may be disposed on the separation layer 250. The second electrode 270 may include a conductive layer 276 and a surface layer 278. The conductive layer 276 and the surface layer 278 may be analogous to the conductive layer 236 and the surface layer 238 of the first electrode 230. The first electrode 230 may be coupled to a voltage source 280 via a first conductor 281 and the second electrode 270 may be coupled to the voltage source 280 via a second conductor 282.

Fluid in the first subchamber 261 may be in contact with the surface layer 238 of the first electrode 230, and fluid in the second subchamber 262 may be in contact with the surface layer 278 of the second electrode 270. In the nucleotide sensing device 202 of FIG. 2, the first subchamber 261 may be a positive chamber (i.e., associated with a trans-electrode) and the second subchamber 262 may be a negative chamber (i.e., associated with a cis-electrode), but it will be understood that the polarity of subchambers 261, 262 may be reversed.

Although the chamber 260 is depicted as a closed chamber, it will be understood that this is optional. For example, the chamber 260 may be an open chamber. Moreover, as will be discussed in greater detail below, the chamber 260 may include enough conductive fluid to fill the first subchamber 261 and cover the second electrode 270.

FIG. 2B generally illustrates a detail of the biological pore 256 of FIG. 2A in accordance with an aspect of the disclosure. As noted above, the biological pore 256 may be embedded in the separation layer 250, and the separation layer 250 may separate the first subchamber 261 from the second subchamber 262.

The biological pore 256 may include, for example, a translocator 258 and an assembler 259. The translocator 258 permits fluid communication (for example, passage of conductive fluid) between the first subchamber 261 and the second subchamber 262. For example, if the second subchamber 262 is negatively charged and the first subchamber 261 is positively charged, then negative ions (for example, Cl⁻) may pass from the second subchamber 262 to the first subchamber 261 via the translocator 258 and/or positive ions (for example, K⁺ and/or H⁺) may pass from the first subchamber 261 to the second subchamber 262. In some implementations, the translocator 258 may include alpha hemolysin.

The assembler 259 may be configured to, for example, separate a double-stranded DNA molecule 290 into a first DNA strand 291 and a second DNA strand 292 and/or combine the first DNA strand 291 and the second DNA strand 292 into the double-stranded DNA molecule 290 (as depicted in FIG. 2B). In some implementations, the assembler 259 may include DNA polymerase.

FIG. 2B generally illustrates the separation and/or combination of a double-stranded DNA molecule 290. For example, the double-stranded DNA molecule 290 may move from the second subchamber 262 into the assembler 259, where it is separated by the assembler 259 into the first DNA strand 291 and the second DNA strand 292. The first DNA strand 291 may be led into the translocator 258 and translocated across the separation layer 250, from the second subchamber 262 to the first subchamber 261. As another example, the first DNA strand 291 may be drawn from the first subchamber 261 through the translocator 258 and into the assembler 259, where it is combined with the second DNA strand 292 into the double-stranded DNA molecule 290. The double-stranded DNA molecule 290 may then be moved into the second subchamber 262.

In some implementations, the following method may be used to perform nucleotide sequencing using the nucleotide sensing device 202 of FIG. 2 and the biological pore 256 of FIG. 2A. First, a voltage may be applied to the first electrode 230 and the second electrode 270 via the first conductor 281 and the second conductor 282, respectively. As a result, a positive charge may appear on the first electrode 230 and a negative charge may appear on the second electrode 270.

As an example, the second electrode 270 may include a surface layer 278 including AgCl and a conductive layer 276 including Ag. When the voltage V is applied (such that the second electrode 270 is negatively charged), the AgCl in the second electrode 270 may be converted into Ag and chlorine electrolytes, i.e., AgCl(s)+e⁻→Ag(s)+Cl⁻. As the second electrode 270 generates Cl⁻ ions, the second subchamber 262 may become negatively charged.

Moreover, the first electrode 230 may include a surface layer 238 including AgCl and a conductive layer 236 including Ag. When the voltage V is applied (such that the first electrode 230 is positively charged), the Ag in the first electrode 230 may combine with Cl⁻ ions in the first subchamber 261, i.e., Ag(s)+Cl⁻→AgCl(s)+e⁻. As the first electrode 230 combines Cl⁻ ions into AgCl, the first subchamber 261 may become positively charged.

As a result, ions in the chamber 260 may have a tendency to flow toward either the first subchamber 261 (which is positively charged) or the second subchamber 262 (which is negatively charged). For example, Cl⁻ ions in the chamber 260 (including Cl⁻ ions generated at the second electrode 270) may have a tendency to flow toward the positively-charged first subchamber 261.

As the first electrode 230 generates electrons e⁻, an electrical current i_{PORE} may flow through the via 222 to the semiconductor device 212.

Because Cl⁻ ions may have a tendency to flow toward the positively-charged first subchamber 261, the Cl⁻ ions may translocate across the separation layer 250 via the biological pore 256. However, the biological pore 256 may also be configured to translocate a strand of nucleotides (for example, the first DNA strand 291, as shown in FIG. 3).

As the first DNA strand 291 shown in FIG. 2B is being translocated, it may impede the flow of Cl⁻ ions through the biological pore 256. As a result, the current i_{PORE} may be reduced due to the translocation of the first DNA strand 291. Moreover, different types of nucleotide may have different effects on the flow of Cl⁻ ions through the biological pore 256.

Accordingly, as different types of nucleotide pass through the biological pore 256, different quantities of Cl⁻ ions may pass through the biological pore 256, and a different electrical current i_{PORE} may be measured at the semiconductor device 212. For example, a C nucleotide may cause a current i_{C}, an A nucleotide may cause a current i_{A}, a T nucleotide may cause a current i_{T}, and a G nucleotide may cause a small current i_{G}. As the first DNA strand 291 passes through the biological pore 256, the nucleotide sensing device 202 will generate a current waveform i_{PORE}(t) that indicates the sequence of nucleotides in the first DNA strand 291.

FIG. 3 generally illustrates a pair 300 of nanopore-based nucleotide sensing devices 301, 302 in accordance with aspects of the disclosure. The nucleotide sensing devices 301, 302 may be analogous to the nucleotide sensing device 112 depicted in FIG. 1 and/or the nucleotide sensing devices 201, 202 depicted in FIG. 2.

The pair 300 of nucleotide sensing devices 301, 302 depicted in FIG. 3 has a number of components in common with the nucleotide sensing devices 201, 202 depicted in FIG. 2A. For example, the substrate 210, the semiconductor device 212, the insulator 220, the via 222, and the first electrode 230 having the adhesion/diffusion layer 234, the conductive layer 236, and the surface layer 238 may be analogous to similarly-numbered components depicted in FIG. 2A. The second electrode 270 having the conductive layer 276 and the surface layer 278 and the voltage source 280, the first conductor 281, and the second electrode 270 may also be analogous to similarly-numbered components depicted in FIG. 2A. For brevity, further description of these components will be omitted here.

Like the nucleotide sensing devices 201, 202 depicted in FIG. 2A, the nucleotide sensing devices 301, 302 depicted in FIG. 3 may include a chamber 260 divided into a first subchamber 261 and a second subchamber 262 by a separation layer 350. The separation layer 350 may comprise a nanopore 355 that permits fluid communication (for example, passage of conductive fluid) between the first subchamber 261 and the second subchamber 262.

Unlike the nucleotide sensing devices 201, 202 depicted in FIG. 2A, the nucleotide sensing devices 301, 302 depicted in FIG. 3 omit the biological pore 256. As will be discussed in greater detail below, the size of the nanopore 355 may be selected to permit passage of a single nucleotide strand. Because different types of nucleotide strands may have different thicknesses, the diameter of a nanopore that is “selected to permit passage of a single nucleotide strand” may differ depending on the type of nucleotide strand that is intended to be passed.

The nucleotide sensing devices 301, 302 depicted in FIG. 3, which omit the biological pore 256, may generate the current waveform i_{PORE}(t) in substantially the same manner as the nucleotide sensing devices 201, 202 depicted in FIG. 2A. In particular, a nucleotide strand may pass through the nanopore 355. As different types of nucleotide pass through the biological pore 256, different quantities of ions may pass through the nanopore 355, and a different electrical current i_{PORE} may be measured at the semiconductor device 212.

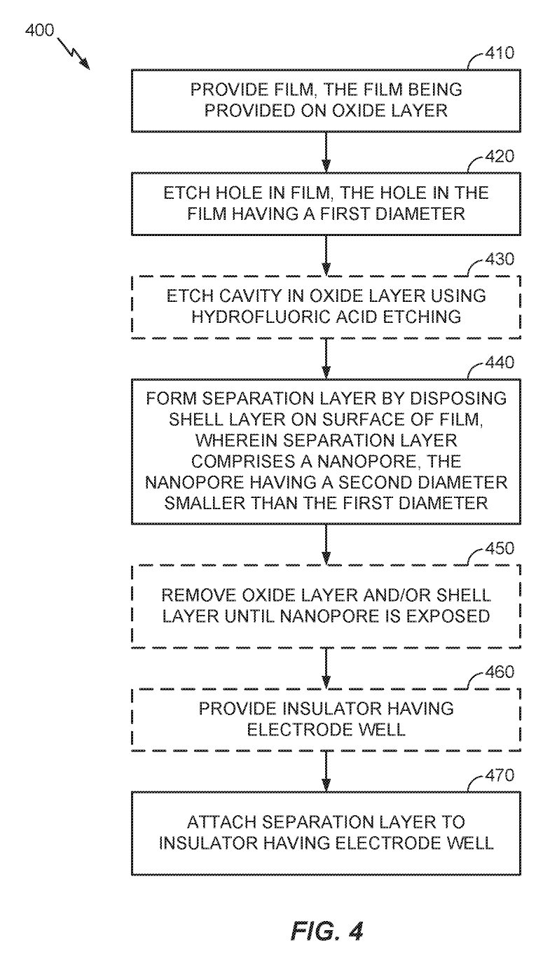

FIG. 4 generally illustrates a method 400 for forming a nucleotide sensing device such as the nucleotide sensing devices 201, 202 of FIG. 2A and/or the nucleotide sensing devices 301, 302 of FIG. 3. For purposes of illustration, the method 400 will be described below as a method of forming the nucleotide sensing device 301 of FIG. 3.

At 410, the method 400 provides a film, the film being provided on an oxide layer. For example, silicon-on-insulator (SOI) technology provides a thin film on an oxide layer. The thin film may include, for example, silicon (Si) and the oxide layer may comprise, for example, silicon dioxide (SiO₂). The film and oxide layer may be provided on a handle layer. The handle layer may provide mechanical support to the film and oxide layer and may comprise, for example, Si.

At 420, the method 400 etches a hole in the film. The hole in the film may have a first diameter. The etching may be performed using photolithography. In some implementations, the minimum value of the first diameter may be limited by a minimum feature size of the photolithographic technique. For example, using a particular photolithographic technique, it may be possible to etch a hole having a first diameter of no less than ten nanometers. As noted above, to perform nanopore sequencing, the presence of a single nucleotide strand within the nanopore disrupts the conduction of the ions, thereby causing a change in the electric current and generation of a nucleotide sensing signal reflecting the particular nucleotides in the nucleotide strand. However, a hole having a first diameter of, for example, ten nanometers will permit simultaneous passage of a plurality of nucleotide strands. Accordingly, a smaller diameter is needed in order to permit passage of a single nucleotide strand. As used herein, the word “hole” may refer to a hole of any shape, for example, circle, ellipse, square, rectangle, polygon, etc.

At 430, the method optionally etches a cavity in the oxide layer. The etching of the cavity at 430 may be performed using any form of etching that selects for removal of the oxide layer. For example, hydrofluoric acid etching may be suitable for removing the oxide layer while leaving the film substantially unaffected. Accordingly, hydrofluoric acid may be permitted to pass through the hole etched at 420 and may therefore be exposed to the oxide layer. The hydrofluoric acid may then dissolve the portions of the oxide layer with which it comes into contact. The dissolution of the oxide layer may occur at a predictable rate, and that size of the cavity may be a function of the duration of exposure time.

At 440, the method 400 forms a separation layer by disposing a shell layer on a surface of the film, wherein the separation layer comprises a nanopore, the nanopore having a second diameter smaller than the first diameter. The separation layer and nanopore formed at 440 may be analogous to the separation layer 350 and nanopore 355 depicted in FIG. 3.

In some implementations, thermal oxidation of the film may be used to form the shell layer on the surface of the film. In particular, the film and/or the film's surrounding environs are provided with heat and oxygen. Over time, the film oxidizes and a shell layer made of oxidized material forms on the film. The formation of the shell layer occurs at a predictable and controllable rate. For example, a film comprising silicon may be oxidizes such that a shell layer of silicon dioxide is formed.

Additionally or alternatively, atomic layer deposition may be used to form the shell layer on the surface of the film. In particular, a precursor gas is added to the film's surrounding environs. Each cycle of precursor gas may result in the deposit of a single layer of solid material to one or more surfaces of the film. For example, each single layer may have a thickness of one nanometer.

To perform the forming at 440, the method 400 may optionally determine that the nanopore 355 has the second diameter, and terminating the forming of the shell layer in response to a determination that the nanopore 355 has the second diameter. In some implementations, the second diameter may be suitable for accommodating a translocator analogous to the translocator 258 depicted in FIG. 2A. For example, the second diameter may be in the range of half a nanometer to two nanometers (1.0-6.0 nm). In other implementations, the second diameter may be suitable for passing exactly one nucleotide strand at a time (as depicted in FIG. 3). For example, the second diameter may be in the range of half a nanometer to two nanometers (0.5-2.0 nm).

In some implementations, the formation of the separation layer may occur at a predictable rate. Accordingly, the amount of time that is necessary to narrow the hole in the film having the first diameter to the nanopore 355 having the second diameter may be predictable. Thus, the termination of the forming of the shell layer at 440 may be responsive to (i) a determination that the forming of the shell layer at 440 has been performed for a predetermined amount of time and (ii) an inference, based on the passage of time, that the hole in the film having the first diameter has narrowed to a nanopore having the second diameter.

At 450, the method 400 may optionally remove the oxide layer and/or the shell layer until the nanopore 355 is exposed from the removal side. For example, as noted above, the film and oxide layer may be provided on a handle layer and the method 400 may optionally include etching of a cavity in the oxide layer. The nanopore may be exposed after the handle wafer is removed and/or a substantial portion of the oxide layer is removed. The removing at 450 may also include removal of portions of the shell layer that have formed on the handle wafer and/or the oxide layer.

At 460, the method 400 optionally provides an insulator having an electrode well. The insulator and electrode well may be analogous to the insulator 220 and first subchamber 261 depicted in FIGS. 2A. In some implementations, the electrode well may be equipped with an electrode analogous to the first electrode 230 depicted in FIG. 2A.

At 470, the method 400 attaches the separation layer 350 to the insulator 220 having the electrode well. The attaching at 470 may be performed, for example, by bonding a portion of a surface of the insulator 220 to a portion of a surface of the separation layer 350.

Although the method 400 is depicted in FIG. 4 as if it is to be performed in a specific order, it will be understood that the method 400 may be performed in any suitable sequence, including sequences other than the sequence depicted in FIG. 4. For example, the providing at 460 and the attaching at 470 may precede the removing at 450.

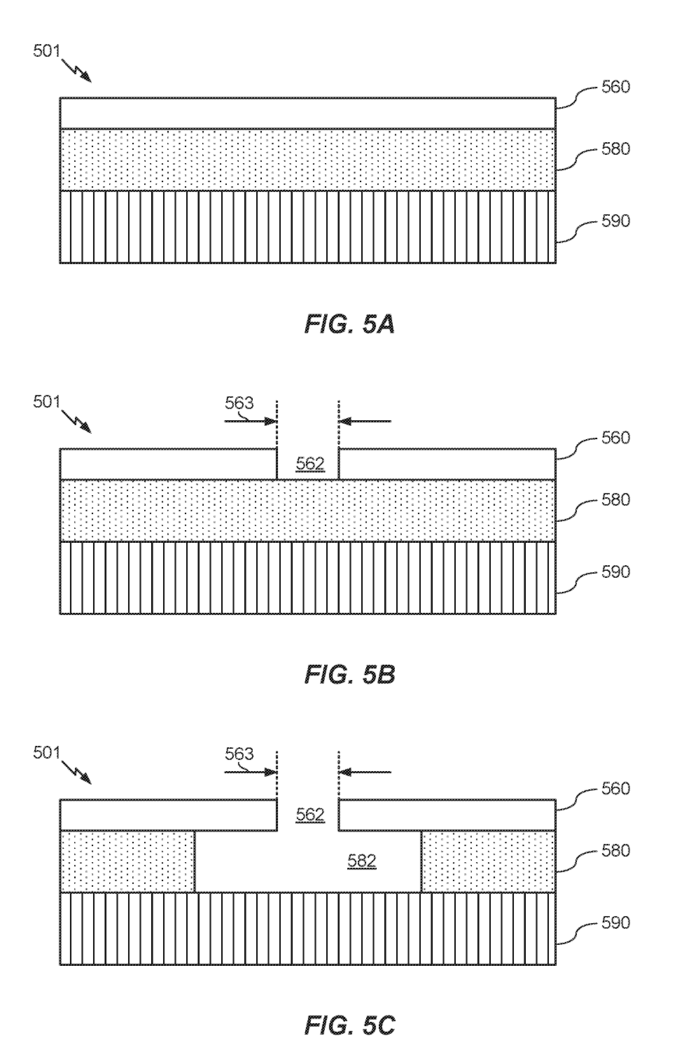

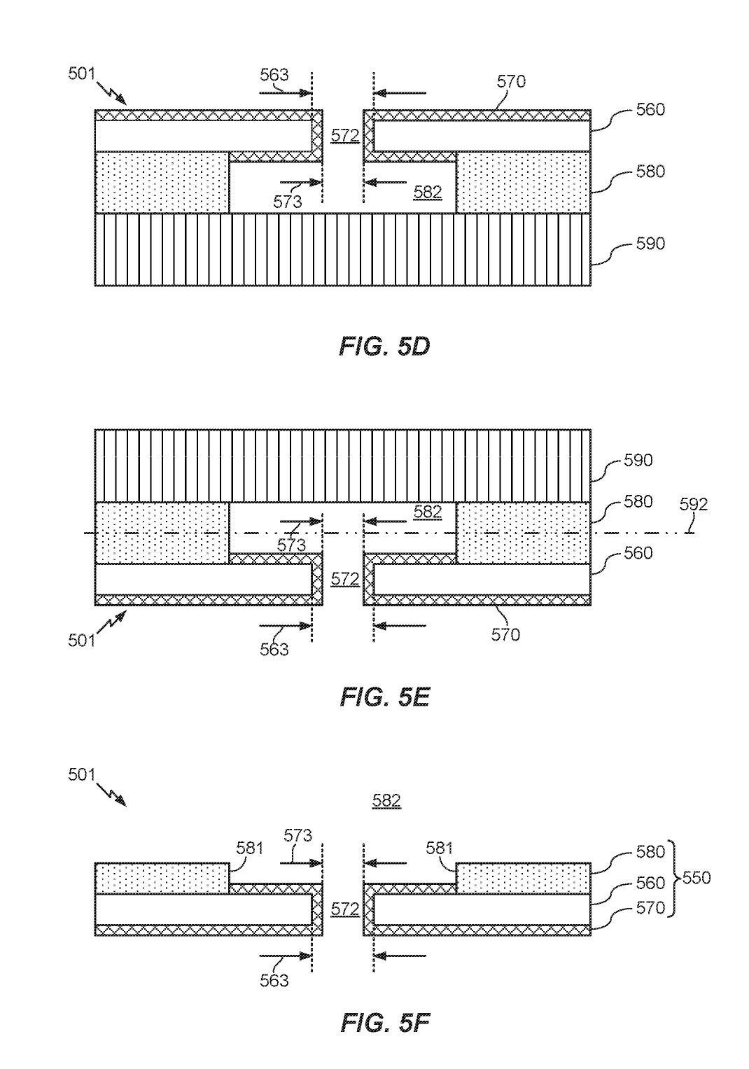

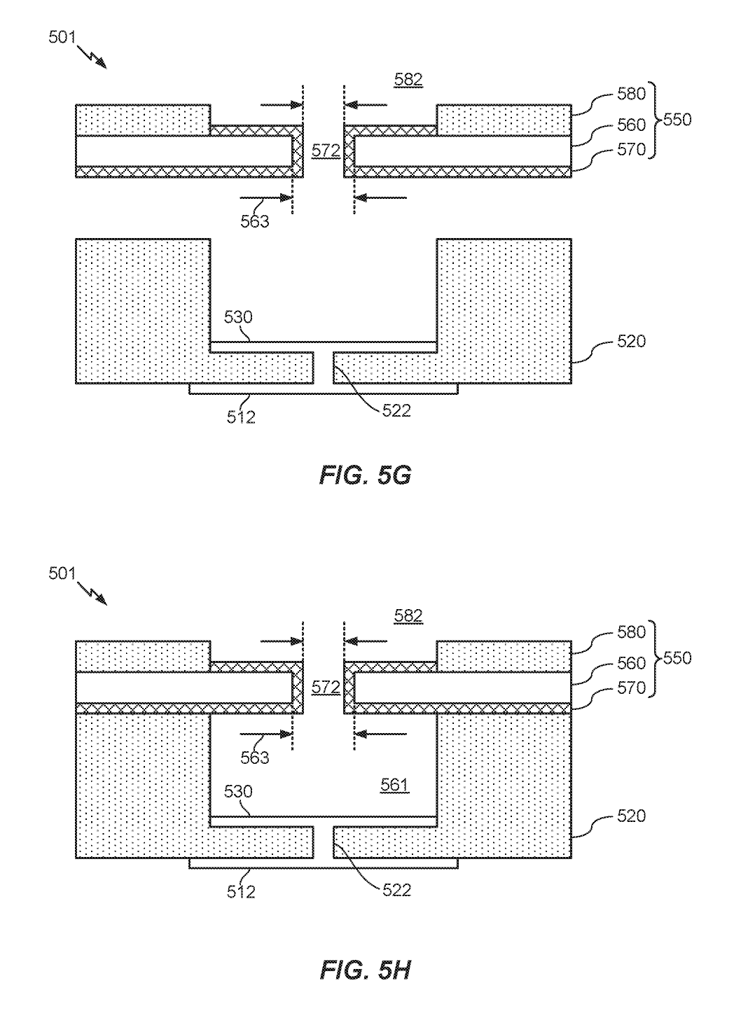

FIGS. 5A-5H generally illustrate a nucleotide sensing device 501 in various stages of fabrication, as will be described in greater detail below. The nucleotide sensing device 501 depicted in FIGS. 5A-5H may be analogous to the nucleotide sensing device 201, 202 of FIG. 2A and/or the nucleotide sensing devices 301, 302 of FIG. 3.

FIG. 5A generally illustrates the nucleotide sensing device 501 in a first stage of fabrication. In the first stage, the nucleotide sensing device 501 comprises a film 560 provided on an oxide layer 580. The oxide layer 580 is provided on a handle layer 590. The providing at 410 (depicted in FIG. 4) may be performed by providing the film 560, the oxide layer 580, and the handle layer 590 as depicted in FIG. 5A. As noted above, such an arrangement may be an SOI arrangement.

FIG. 5B generally illustrates the nucleotide sensing device 501 in a second stage of fabrication. A hole 562 is provided in the film 560. The hole 562 has a hole diameter 563. The etching at 420 (depicted in FIG. 4) may be performed by etching the hole 562 in the film 560 as depicted in FIG. 5A. The hole diameter 563 may be analogous to the first diameter referred to in FIG. 4.

FIG. 5C generally illustrates the nucleotide sensing device 501 in a third stage of fabrication. An oxide cavity 582 is provided in the oxide layer 580. The etching at 430 (depicted in FIG. 4) may be performed by providing the oxide cavity 582 in the oxide layer 580 as depicted in FIG. 5A.

FIG. 5D generally illustrates the nucleotide sensing device 501 in a fourth stage of fabrication. In particular, a shell layer 570 is formed on the film 560. The forming at 440 (depicted in FIG. 4) may be performed by forming the shell layer 570 on the film 560 as depicted in FIG. 5D. As noted above, the forming of the shell layer 570 may be performed using, for example, thermal oxidation and/or atomic layer deposition. In some implementations, the shell layer 570 may also form on the oxide layer 580, the handle layer 590, or any combination thereof. However, the additional formation of the shell layer 570 is omitted from FIGS. 5A-5H for clarity of illustration. As will be understood from a comparison of FIG. 5D to FIG. 5C, the hole 562 in the film 560 (having the hole diameter 563) has been narrowed due to the forming of the shell layer 570. In particular, a nanopore 572 having a nanopore diameter 573 has been formed.

The nanopore diameter 573 may be analogous to the second diameter referred to in FIG. 4. As noted above, the second diameter may be suitable for passing exactly one nucleotide strand at a time. Moreover, the formation of the shell layer 570 may occur at a predictable rate. Accordingly, the transition from the nucleotide sensing device 501 depicted in FIG. 5C (having the hole 562 with hole diameter 563) to the nucleotide sensing device 501 depicted in FIG. 5D (having the nanopore 572 with nanopore diameter 573) may be predictable. Thus, the termination of the forming of the shell layer 570 may be responsive to (i) a determination that the forming of the shell layer 570 has been performed for a predetermined amount of time and (ii) an inference, based on the passage of time, that the hole 562 in the film 560 having the hole diameter 563 has narrowed to a nanopore 572 having the nanopore diameter 573.

FIG. 5E generally illustrates the nucleotide sensing device 501 in a fifth stage of fabrication. In particular, the nucleotide sensing device 501 has flipped along a horizontal axis. It will be understood that the nucleotide sensing device 501 depicted in FIG. 5E may not be structurally distinct from the nucleotide sensing device 501 depicted in FIG. 5D, and has merely been flipped for illustrative purposes. However, FIG. 5E further depicts a removal boundary 592 that is relevant to the sixth stage of fabrication (described below).

FIG. 5F generally illustrates the nucleotide sensing device 501 in a sixth stage of fabrication. The handle layer 590 has been removed. Moreover, a portion of the oxide layer 580 has been removed, in particular, a portion of the oxide layer 580 that was beyond the removal boundary 592 depicted in FIG. 5E. The removing at 450 (depicted in FIG. 4) may be performed by removing at least the handle layer 590 as depicted in FIG. 5F. It will be understood that the exact position of the removal boundary 592 may be arbitrarily selected. However, the removal boundary 592 may be selected such that the nanopore 572 is exposed upon removal.

As noted above, FIG. 5D depicts the shell layer 570 as having been formed exclusively on the film 560. However, in the event that the shell layer 570 forms on the oxide layer 580 and/or the handle layer 590 as well, then portions of the shell layer 570 may be disposed beyond the removal boundary 592. Accordingly, these portions of the shell layer 570 that are disposed beyond the removal boundary 592 may be removed as well.

The removal of the handle layer 590, portions of the oxide layer 580, portions of the shell layer 570, or any combination thereof, may be performed with any suitable process, for example, polishing or chemical etching. The remaining portions depicted in FIG. 5F may constitute a separation layer 550 analogous to the separation layer 250 depicted in FIGS. 2A and 3.

As will be understood from FIG. 5E, the removal boundary 592 may be selected such that it splits the oxide cavity 582. One advantage of this technique is that the remaining portions of the oxide layer 580 form a ridge 581 that surrounds the nanopore 572. Once the nucleotide sensing device 501 is in operation, the ridge 581 may serve to funnel ions and/or nucleotide strands toward the nanopore 572.

In other implementations, the removal boundary 592 may be set such that it is flush with the top surface of the oxide cavity 582 (such that the height of the ridge 581 is increased) or flush with the bottom surface of the oxide cavity 582 (such that the height of the ridge 581 is diminished to zero).

FIG. 5G generally illustrates the nucleotide sensing device 501 in a seventh stage of fabrication. In particular, a semiconductor device 512, an insulator 520 having a via 522, and an electrode 530 have been provided. The providing at 460 (depicted in FIG. 4) may be performed by providing the semiconductor device 512, the insulator 520, and the electrode 530 as depicted in FIG. 5G.

FIG. 5H generally illustrates the nucleotide sensing device 501 in an eighth stage of fabrication. In particular, the separation layer 550 has been attached to a surface of the insulator 520. The attaching at 470 (depicted in FIG. 4) may be performed by attaching the separation layer 550 to the insulator 520 as depicted in FIG. 5G. After attaching has been performed, a first electrode well 561 may be formed. The first electrode well 561 may be analogous to the first subchamber 261 depicted in FIGS. 2A and 3. A second electrode well analogous to the second subchamber 262 depicted in FIGS. 2A and 3 may include portions of the oxide cavity 582. The nanopore 572 may permit fluid communication into (or out of) the first electrode well 561.

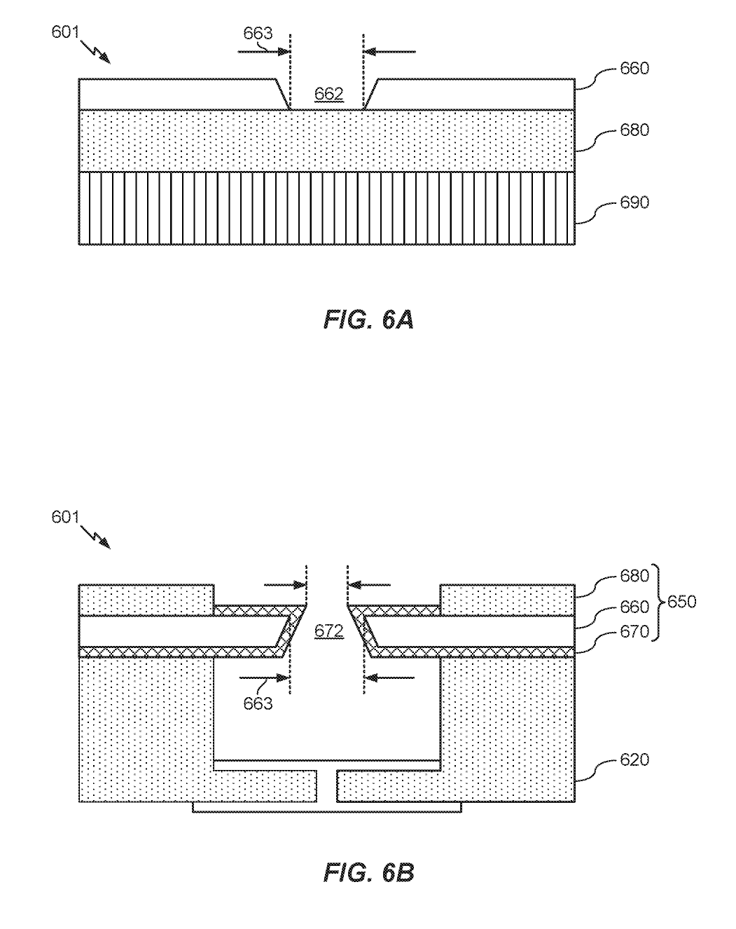

FIGS. 6A-6B generally illustrate a nucleotide sensing device 601 in various stages of fabrication, as will be described in greater detail below. The nucleotide sensing device 601 depicted in FIGS. 6A-6B may be analogous in some respects to the nucleotide sensing device 501 depicted in FIGS. 5A-5H.

FIG. 6A generally illustrates the nucleotide sensing device 601 in a stage of fabrication analogous to the second stage of fabrication depicted in FIG. 5B. Analogous to the nucleotide sensing device 501, the nucleotide sensing device 601 comprises a film 660, an oxide layer 680, and a handle layer 690. The nucleotide sensing device 601 also comprises a hole 662. Although the hole 662 may be analogous to the hole 562 in some respects, it will be understood that the hole 662 is a conical hole or a tapered hole. A hole diameter 663 may be defined as a diameter of the hole 662 at its narrowest.

FIG. 6B generally illustrates the nucleotide sensing device 601 in a stage of fabrication analogous to the eighth stage of fabrication depicted in FIG. 5H. Analogous to the nucleotide sensing device 501, the nucleotide sensing device 601 comprises a insulator 620 to which a separation layer 650 (comprising the film 660, the oxide layer 680, and a shell layer 670) is attached. The nucleotide sensing device 601 also comprises a nanopore 672. Although the nanopore 672 may be analogous to the nanopore 572 in some respects, it will be understood that the nanopore 672 is a conical nanopore. A nanopore diameter 573 may be defined as a diameter of the nanopore 672 at its narrowest.

It will be understood that various components described herein may constitute means for performing various functions. For example, the insulator 220 depicted in FIG. 2A may constitute a means for insulating. The separation layer 250 depicted in FIG. 2A and/or the separation layer 550 depicted in FIG. 5F may constitute a means for separating. The means for separating may include (i) means for supporting the means for separating and (ii) means for covering the means for supporting. The film 560 depicted in FIGS. 5A-5F may constitute the means for supporting the means for separating. The shell layer 570 depicted in FIGS. 5D-5F may constitute means for covering the means for supporting. The semiconductor device 212 may constitute a means for sensing. The first electrode 230 may constitute a first means for contacting and the second electrode 270 may constitute a second means for contacting.

While the foregoing disclosure shows various illustrative aspects, it should be noted that various changes and modifications may be made to the illustrated examples without departing from the scope defined by the appended claims. The present disclosure is not intended to be limited to the specifically illustrated examples alone. For example, unless otherwise noted, the functions, steps, and/or actions of the method claims in accordance with the aspects of the disclosure described herein need not be performed in any particular order. Furthermore, although certain aspects may be described or claimed in the singular, the plural is contemplated unless limitation to the singular is explicitly stated.

What is claimed is:

1. A nucleotide sensing device, comprising:
a insulator having an electrode well; and
a separation layer attached to the insulator, the separation layer including a film and a shell layer;
wherein:
the film has a hole, the hole having a first diameter;
the shell layer is disposed on a surface of the film, and at least a portion of the shell layer is disposed within the hole; and
the separation layer comprises a nanopore, the nanopore permitting fluid communication across the separation layer, the nanopore being disposed within the hole and having a second diameter smaller than the first diameter.

2. The nucleotide sensing device of claim 1, wherein the film is formed of silicon.

3. The nucleotide sensing device of claim 1, wherein the first diameter is suitable for simultaneously passing a plurality of nucleotide strands.

4. The nucleotide sensing device of claim 1, wherein:
the hole comprises a conical hole, the first diameter being a diameter of the conical hole at its narrowest; and
the nanopore is a conical nanopore, the second diameter being a diameter of the conical nanopore at its narrowest.

5. The nucleotide sensing device of claim 1, wherein the separation layer further comprises an oxide layer, the oxide layer further comprising a cavity.

6. The nucleotide sensing device of claim 1, wherein the shell layer is formed using:
thermal oxidation of the film;
atomic layer deposition on the film; or
any combination thereof.

7. The nucleotide sensing device of claim 1, wherein the second diameter is suitable for passing exactly one nucleotide strand at a time.

8. The nucleotide sensing device of claim 1, wherein the nanopore is in fluid communication with the electrode well.

9. The nucleotide sensing device of claim 1, wherein the electrode well is a first electrode well having a first electrode disposed therein, the first electrode being coupled to a complementary metal-oxide-semiconductor circuit.

10. The nucleotide sensing device of claim 9, further comprising a second electrode well having a second electrode disposed therein, wherein the second electrode is coupled to the complementary metal-oxide-semiconductor circuit.

11. A method of forming a nucleotide sensing device, comprising:
providing a film, the film being provided on an oxide layer;
etching a hole in the film, the hole in the film having a first diameter;
forming a separation layer by disposing a shell layer on a surface of the film, wherein the separation layer comprises a nanopore, the nanopore having a second diameter smaller than the first diameter; and
attaching the separation layer to a insulator having an electrode well.

12. The method of claim 11, wherein the film is formed of silicon.

13. The method of claim 11, wherein etching of the hole comprises photolithographic etching of the hole, the first diameter being suitable for simultaneously passing a plurality of nucleotide strands.

14. The method of claim 11, wherein:
etching of the hole comprises etching a conical hole, the first diameter being a diameter of the conical hole at its narrowest; and
the nanopore is a conical nanopore, the second diameter being a diameter of the conical nanopore at its narrowest.

15. The method of claim 11, wherein the separation layer further comprises an oxide layer, the method further comprising:
prior to the disposing of the shell layer on the surface of the film, etching a cavity in the oxide layer using hydrofluoric acid etching.

16. The method of claim 11, wherein forming of the shell layer comprises:
thermal oxidation of the film;
atomic layer deposition on the film; or
any combination thereof.

17. The method of claim 11, further comprising:
determining that the nanopore has the second diameter, the second diameter being suitable for passing exactly one nucleotide strand at a time; and
terminating the forming of the shell layer in response to a determination that the nanopore has the second diameter.

18. The method of claim 11, wherein attaching the separation layer to the insulator comprises:
attaching the separation layer to the insulator such that the nanopore is in fluid communication with the electrode well.

19. The method of claim 18, further comprising:
removing the oxide layer and/or the shell layer until the nanopore is exposed.

20. The method of claim 19, wherein the electrode well is a first electrode well having a first electrode disposed therein, the first electrode being coupled to a complementary metal-oxide-semiconductor circuit, and the method further comprises:
providing a second electrode well having a second electrode disposed therein, the second electrode being coupled to the complementary metal-oxide-semiconductor circuit.

21. A nucleotide sensing device, comprising:
means for insulating; and
means for separating attached to the means for insulating, the means for separating including means for supporting the means for separating and means for covering the means for supporting;
wherein:
the means for supporting has a hole, the hole having a first diameter;
the means for covering is disposed on a surface of the means for supporting, and at least a portion of the means for covering is disposed within the hole; and
the means for separating comprises means for permitting fluid communication across the means for separating, the means for permitting fluid communication being disposed within the hole and having a second diameter smaller than the first diameter.

22. The nucleotide sensing device of claim 21, wherein the means for supporting is formed of silicon.

23. The nucleotide sensing device of claim 21, wherein the first diameter is suitable for simultaneously passing a plurality of nucleotide strands.

24. The nucleotide sensing device of claim 21, wherein:
the hole comprises a conical hole, the first diameter being a diameter of the conical hole at its narrowest; and
the means for permitting fluid communication is a conical nanopore, the second diameter being a diameter of the conical nanopore at its narrowest.

25. The nucleotide sensing device of claim 21, wherein the means for separating further comprises an oxide layer, the oxide layer further comprising a cavity.

26. The nucleotide sensing device of claim 21, wherein the means for covering is formed using:
thermal oxidation of a film;
atomic layer deposition on the film; or
any combination thereof.

27. The nucleotide sensing device of claim 21, wherein the second diameter is suitable for passing exactly one nucleotide strand at a time.

28. The nucleotide sensing device of claim 21, wherein the means for permitting fluid communication is in fluid communication with an electrode well, the electrode well being included in the means for insulating.

29. The nucleotide sensing device of claim 28, wherein the electrode well is a first electrode well having a first means for contacting disposed therein, the first means for contacting being coupled to means for sensing.

30. The nucleotide sensing device of claim 29, further comprising a second electrode well having a second means for contacting disposed therein, wherein the second means for contacting is coupled to the means for sensing.
